(19) United States
(12) Patent Application Publication
(10) Pub. No.: US 2007/0135877 A1
(43) Pub. Date: Jun. 14, 2007

(54) USE OF MICROWAVE ENERGY FOR THERMOTHERAPY

(75) Inventor: Frank G. Pringle, Medford, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Mobilestream Oil, Inc., West Berlin, NJ (US)

(21) Appl. No.: 11/610,962

(22) Filed: Dec. 14, 2006

Related U.S. Application Data
(60) Provisional application No. 60/750,098, filed on Dec. 14, 2005.

(51) Int. Cl. A61F 2/00

(52) U.S. Cl. 607/101

(57) ABSTRACT

Provided are systems and methods that employ microwave energy to selectively destroy damaged or diseased tissue such as tumors in the prostate, brain, breast, and other anatomical locations.

### CROSS-REFERENCE TO RELATED APPLICATIONS

This application claims benefit of U.S. Provisional Application No. 60/750,098, filed Dec. 14, 2005, the disclosure of which is hereby incorporated by reference in its entirety.

### FIELD OF THE INVENTION

The present invention relates to methods and devices for using microwave radiation and more particularly, to methods and devices for treating diseased tissue.

### BACKGROUND OF THE INVENTION

A traditional approach to treatment or elimination of diseased tissue has included surgical excision, a necessarily invasive process that is often accompanied by undesired structural or cosmetic consequences. As contrasted with partial or comprehensive removal of tissue, e.g., mastectomy in the case of breast cancer, recent trends in the management of cancerous growth and other disease conditions have moved toward tissue conservation. See, e.g., Singletary ES, Semin Surg Oncol 2001;20:246-50 (minimally invasive treatment of breast cancer).

For example, ablative techniques are now being applied to the treatment of primary breast tumors, perhaps offering an alternative to surgical excision. Several techniques have been identified as available for treatment of diseased tissue in situ, including radiofrequency ablation, cryoablation, interstitial laser ablation, microwave thermotherapy, and focused ultrasound ablation. Some of these technologies employ probes for delivery of energy for ablating tumors and for monitoring the effect that can be placed precisely within diseased tissue.

Thermal destruction of diseased tissue can comprise the application of either heat or cold. Cryoablation involves the use of a liquid-nitrogen cooled needle, while heating techniques include placing probes in the vicinity of the target tissue to conduct radiofrequency irradiation or laser light energy. Interstitial laser photocoagulation (Dowlatshashi K et al. Am J Surg 2002; 184:359-63) and radiofrequency-induced coagulation (Izzo F et al. Cancer 2001; 92:2036-44) have also been proposed for the treatment of early breast carcinoma. Two additional techniques, focused ultrasound and focused microwave thermotherapy, are truly non-invasive or minimally invasive in that they potentially do not involve any skin puncture.

Using temperatures in the range of at least 45° C. to 53° C., the cytotoxic effects of hyperthermia have been demonstrated in vitro on a variety of cell types. Gerhard H et al., Cancer Therapy by Hyperthermia and Radiation. Streffer C., ed. Baltimore: Urban & Schwartzenberg, 1978:201-3; Giovanella B C et al., Cancer Res 1976; 36:3944-50. Tumoricidal effects have resulted from heating at 43° C. for 60 minutes, with the period of time required to kill tumor cells decreasing by a factor of 2 for each degree increase above this temperature. Id. Such heating was also shown to preferentially kill tumor cells over normal tissue. Id.

Other studies have demonstrated that elevating the temperature of human cells by about 10° F. or more above normal body temperature can result in cell death, including tumor cell death. See Vargas H I et al., Ann Surg Oncol. 2004 February; 11(2):139-46.

Microwave energy is effective in heating high-water content tissue, including cancerous tumors. By concentrating microwave energy, it is possible selectively to heat high-water content disease tissue while leaving healthy tissue unaffected. The temperature of the targeted tissue can be elevated rapidly, eventually exposing the targeted tissue to cytotoxic temperatures. The technique of exposing damaged or diseased tissue to lethal temperatures is known as thermotherapy.

Microwave energy has been viewed as potentially promising due to its ability to preferentially heat high water content tissue, such as breast carcinomas, as compared to relatively low water content adipose and glandular tissues. Chaudhary S S et al., Indian J Biochem Biophys 1984; 21:76-9.

Dr. Alan J. Fenn at the Massachusetts Institute of Technology's Lincoln laboratory developed a concept for heating deep tumors by means of adaptive microwaves, which adjusted to the properties of a patient's tissue in order to concentrate microwave energy at the tumor's position in space. The adaptive microwaves were generated by multiple microwave antennae, collectively referred to as an adaptive phased array, that were inserted beneath the skin in order to direct concentrated microwave energy onto the target tissue. Dr. Fenn's work is described in the Vargas et al. publication (cited previously), which describes how the investigators used a two-channel 915 MHz focused microwave adaptive phased array thermotherapy system and a percutaneous sensor catheter to produce a focused microwave field in the breast to heat and destroy high water content tumor tissue. Vargas H I et al., Ann Surg. Oncol. 11(2):139-146 (2004). The study observed pathologic necrosis in 68% of 25 test subjects, with the degree of coagulative tumor cell necrosis ranging from 25% of the total cancerous complement to 99.9% (observed in one patient). Vargas et al. also provided a statistical model that predicted 100% tumor cell death when a thermal dosage of 209.8 cumulative equivalent minutes (CEM) and a peak tumor temperature of 49.7° C. are achieved. However, the study observed that in patients experimentally assigned to a 120 CEM dose, 47% experienced pain, 27% developed skin erythema, 33% developed edema of the breast or areola, and 13% developed skin thermal burns, suggesting that the thermal dosage expected (based on the statistical mode) to produce 100% efficacy—209.8 CEM—could produce an unacceptable frequency of adverse events. Vargas et al. also recognized the importance among existing techniques of accurate placement of percutaneous microwave-emitting probes in achieving successful percutaneous tumor ablation.

Methods and systems for treating cancerous breast tissue using various means of energetic excitation are disclosed by Hung et al. in U.S. Pat. No. 6,712,816. Hung et al. provides that a fluid or material with a resonance frequency of that of an electromagnetic source, such as radiofrequency and microwave, may be administered to a breast milk duct, followed by application of the electromagnetic energy by introducing an energy delivering tool into the breast duct, to which energy the fluid or material would respond by exhibiting resonating activity and emitting thermotherapeutic heat. Hung et al. specifies that “[m]etallic fluids such as gold or silver colloid” can be used (col. 5, lines 35-37), and that the power supply component of the invention will typically provide energy “in the range of from about 200 kHz to 4 MHz” (col. 11, lines 60-62). Other fluids or materials for use as the target of the electromagnetic energy are not disclosed, nor are other specific electromagnetic frequencies for use in causing excitation of the target fluid or material. Although Hung et al. provides that the resonant energy can also be applied externally to the outside of the whole breast, the patent does not teach the administration of fluids or materials for use as targets of the electromagnetic energy to any other region of the breast, or to other parts of the body in general, than the breast ducts (see, e.g., col. 13, lines 39-47).

Although minimally invasive technologies for the microwave ablation of pathological tissue have been the recent subject of investigation, the field is still under development and requires as-yet undiscovered refinements before clinical deployment can become a widespread reality.

### SUMMARY OF THE INVENTION

The present application is directed to systems and methods that employ microwave energy to selectively destroy tissue such as tumors in the prostate, brain, breast, and other anatomical locations.

The present invention provides methods for microwave treatment of tissue comprising contacting the tissue with particles comprising carbon, and subjecting the tissue to microwave radiation. Also provided are methods for microwave treatment of tissue comprising contacting the tissue with material having a resonating frequency in the range of from about 4 GHz to about 12 GHz, and subjecting the tissue to microwave radiation characterized as having at least one frequency component that corresponds to the resonating frequency of the material.

There are also disclosed systems for microwave treatment of tissue comprising a microwave radiation generator capable of generating microwave radiation characterized as having at least one frequency component in the range of from about 4 GHz to about 12 GHz, said generator being operatively connected to at least one antenna, each of the at least one antenna being capable of transmitting microwave radiation to a tissue region, and comprising a source of particles, wherein the particles are capable of absorbing at least a portion of the transmitted microwave radiation, and are capable of being placed into or near the tissue region.

### BRIEF DESCRIPTION OF THE DRAWINGS

FIG. 1 provides a representation of the inventive system for microwave treatment of tissue during operation.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions, or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a carbon-containing inorganic precursor” is a reference to one or more of such precursors and equivalents thereof known to those skilled in the art, and so forth. When values are expressed as approximations, by use of the antecedent “about,” it will be understood that the particular value forms another embodiment. Where present, all ranges are inclusive and combinable.

The present application is directed to systems and methods that employ microwave energy to selectively heat and destroy tissue such as tumors in the prostate, brain, breast, and other anatomical locations. The present invention utilizes a specialized range of microwave frequencies to selectively heat carbon particles that have been dispersed within a region of tissue, thereby raising the temperature of the cells within the tissue to cytotoxic levels, and represents a minimally invasive therapy that can be performed without sedation or general anesthesia and on an outpatient basis.

It has been discovered that microwave radiation in the frequency range of from about 4 GHz to about 12 GHz is useful for selectively heating materials that can be introduced proximally to damaged or diseased tissue, such as that affected with cancerous or precancerous growth. It has further been found that such materials can comprise carbon particles that absorb energy when irradiated with microwave radiation, while tissue that is distally located from the carbon particles does not absorb the energetic contribution of the microwave radiation. The heat from the energized carbon particles is released to the adjacent diseased tissue, and when sufficient heat is released, the diseased tissue is destroyed. The healthy tissue is left unaffected. Unlike the prior art, the present discovery discloses a particular range of frequencies that is efficacious for the electromagnetic stimulation and heating of carbon particles. Also unlike the prior art, the present invention is also compatible with the treatment of any anatomical location in which diseased tissue may be contacted with carbon particles, and is not limited to the use of, for example, a microwave-emitting probe that can be inserted into a patient's breast milk duct.

Disclosed are methods for microwave treatment of tissue comprising contacting the tissue with particles comprising carbon, and subjecting the tissue to microwave radiation. Also disclosed are methods for microwave treatment of tissue comprising contacting the tissue with material having a resonating frequency in the range of from about 4 GHz to about 12 GHz, and subjecting the tissue to microwave radiation characterized as having at least one frequency component that corresponds to the resonating frequency of the material. As used herein, carbon particles or material having a resonating frequency corresponding to the applied microwave radiation frequency are collectively referred to as “resonating material”.

In preferred embodiments of the disclosed methods, the microwave radiation is a pre-selected microwave radiation frequency. Preferably, the pre-selected microwave radiation frequency will be the resonating microwave frequency, i. e., the microwave radiation frequency at which the particles comprising carbon absorb a maximum amount of microwave radiation. It has been determined that different compositions of the present invention will absorb more or less microwave radiation, depending on the frequency of the microwave radiation applied. It has also been determined that the frequency at which maximum microwave radiation is absorbed differs by composition. By using methods known in the art, a composition of the present invention can be subjected to different frequencies of microwave radiation and the relative amounts of microwave radiation absorbed can be determined. Preferably, the microwave radiation selected is the frequency that comparatively results in the greatest amount of microwave radiation absorption. In one embodiment, the pre-selected microwave radiation frequency is characterized as having at least one frequency component in the range of from about 4 GHz to about 12 GHz. In other embodiments, the pre-selected microwave radiation frequency is characterized as having at least one frequency component in the range of from about 5 GHz to about 9 GHz, from about 6 GHz to about 8 GHz, or from about 6.5 GHz and about 7.5 GHz.

The particles comprising carbon are preferably carbon substances that have a resonating microwave frequency of from about 4 GHz to about 12 GHz. Many forms of carbon are known by those skilled in the art, and, while not intending to exclude other carbon types, it is contemplated that any form of carbon having a resonating microwave frequency of from about 4 GHz to about 12 GHz will be within the scope of the present invention. For example, the particles comprising carbon can comprise carbon black. Carbon black may be described as a mixture of incompletely-burned hydrocarbons, produced by the partial combustion of natural gas or fossil fuels.

Carbon blacks have chemisorbed oxygen complexes (e.g., carboxylic, quinonic, lactonic, phenolic groups and others) on their surfaces to varying degrees depending on the conditions of manufacture. These surface oxygen groups are collectively referred to as the volatile content. In preferred embodiments, the present invention uses carbon black having a moderate volatile content, for example, having about C20 or about C30 volatile constituents. Carbon blacks having a different volatile content are also contemplated as being within the scope of the present invention.

The constituent parts of the resonating material preferably have characteristic dimensions in the micrometer range, although other particle or fragment sizes may also be used. Because carbon particles or particles comprising another resonating material for use in the present invention can be present in numerous configurations, and can be irregular in shape, the term “characteristic dimensions” is used herein to describe the long axis in the case of substantially cylindrical or otherwise oblong particles, and to describe diameter in the case of substantially spherical particles, etc. In preferred embodiments wherein the wherein the carbon particles comprise carbon black, the particles can have characteristic dimensions of about 100 μm. In other embodiments, the particles can have characteristic dimensions of about 10 nm to about 500 μm, of about 100 nm to about 100 μm, or of about 200 nm to about 10 μm.

Preferred are resonating materials having characteristic dimensions that are conducive to ready dispersion within delivery media. Although the resonating materials can be contacted with the tissue by directly introducing the resonating materials into the tissue environment, a more preferred method involves mixing the materials into a delivery medium, which is itself introduced into the tissue environment and contacted with the target tissue. Suitable delivery media can be liquid, in the form of solutions, suspensions, emulsions, syrups, elixirs, and the like. The selected delivery medium should be compatible with the human internal corporeal environment, and to this end aqueous solutions are a preferred delivery medium, one highly favored example being saline. It has been discovered that carbon black particles are readily dispersed within a saline delivery medium, which in turn is readily introduced into the human body. For example, it has been discovered that 30 cc of saline/carbon black solution comprising 40 weight percent carbon black provides favorable results.

In the present invention, in order to effect the step of contacting the tissue with resonating materials, the materials should be somehow introduced into proximity with the tissue, whether such tissue is on or near the surface of the subject's body or at some internal location. In many cases, the damaged or diseased tissue will reside within the subject's body, and in such instances the resonating materials should be applied internally. Although any means of introducing the resonating materials into the subject to the tissue in a safe, and minimally-invasive manner would be suitable for use with the present methods, injection represents a preferred method of transferring the resonating materials into the patient so that they contact the target tissue. As used herein “contacting” means causing the material that will be subjected to the microwave radiation, e.g., carbon particles, to be spatially situated such that the heat released therefrom thermotherapeutically affects the target tissue. Therefore, the preferred method of injecting the resonating material involves causing the resonating material to be situated within or near the diseased tissue.

Prior to microwave irradiation of the tissue, or even prior to contacting the tissue with the carbon particles or material having a resonating frequency corresponding to the applied microwave radiation frequency, practitioners may wish to localize a target area of the tissue, so that, for example, a specific area of the tissue can be designated for contacting with resonating material, post-contacting irradiation, or both. The disclosed methods may therefore further comprise identifying a target area of the tissue. Once a target area of the tissue has been identified, it can be contacted with resonating material that is precisely delivered into or near the identified area, or subjected to focused microwave radiation that has been directed particularly to that area. The target area, which can be a tumor mass or any other area of particular interest, can be identified using an imaging device. The imaging device may be selected from any suitable apparatus, including an ultrasound device, a magnetic resonance imaging device, a fluoroscopy device, a positron emission tomography device, a tomography device, a radiological device, or the like. Those skilled in the art will be familiar with the operation of an imaging device to identify a target area of the tissue.

In some instances, the application of the resonating material to the tissue will be followed by a period of time during which no further steps are performed, in order to permit the dispersion of the resonating material within the target tissue. The subject into whom the resonating material has been introduced will be instructed to wait for a specific period of time before initiation of the next treatment step. For example, a period of from about a few minutes up to about a few hours is sufficient to permit the resonating material to disperse within the target tissue. For example, if injection is the selected means of transferring the resonating material into the subject, then following injection, the subject can be asked to wait for a certain period of time and then to return to the treatment facility so that the next stages of treatment can be commenced. In some embodiments of the present invention, the period of time will be about 10 minutes. In other instances, the period of time will be up to one hour, or up to two hours or more.

Once the resonating material is contacted with the tissue, the tissue is subjected to microwave radiation, thereby also causing the resonating material to be subjected to microwave radiation. The microwave radiation energetically stimulates the resonating material, causing the particles or material to heat up, some of which heat in turn being transferred to the tissue. In some embodiments of the present invention, the tissue is subjected to microwave radiation for a period of time that is sufficient to raise the temperature of the tissue by at least about 5° F. above the temperature of the tissue prior to the starting temperature of the tissue, i.e., the temperature of the tissue prior to the application of microwave radiation thereto. Under typical circumstances, when the subject is a person, the starting temperature of the tissue will be normal human body temperature, or 98.6° F./37° C. Preferably, the tissue is subjected to microwave radiation for a period of time sufficient to cause the temperature of the tissue to be raised by at least about 10° F. above the starting temperature of the tissue. The tissue can also be subjected to microwave radiation for a period of time sufficient to raise the temperature of the tissue up to about 10° F. above the temperature of the tissue prior to the application of microwave radiation thereto. In the instant invention, the approximate irradiation time necessary to complete a full course of treatment can be about 30 minutes. However, depending on numerous factors, including the power of the microwave generator, and the distance of the tissue from the skin surface and from the microwave antennas, the irradiation time can be shorter or longer than 30 minutes.

The inventive methods can further comprise placing a temperature probe into or near the target tissue, so that the change in the tissue's temperature during microwave irradiation can be monitored, and the irradiation discontinued once the desired temperature change has been attained. The temperature probe may be subcutaneously introduced into or near the tissue, or may make use of temperature-monitoring techniques that do not require subcutaneous insertion. Other means of monitoring tissue temperature, such as infrared, can also be employed.

The microwave radiation can be delivered to the tissue using one or more microwave antennas. In preferred embodiments of the instant invention, the microwave radiation is delivered using three microwave antennas. Where three microwave antennas are used, the microwave radiation can be directed onto the tissue or a specific portion thereof (a “target area”) from each of the antennas. In such instances, it will be possible to adjust the directionality of one or more of the antennas so that the microwave radiation emitted therefrom will be directed onto the desired target area, which allows the concentration or focusing of the microwave radiation onto the chosen situs. One method of adjusting the antennas so that each one is focused onto the target area is triangulation, which can be accomplished manually or with the assistance of a machine, such as a computer. Computer-assisted triangulation represents a preferred method of adjusting the three microwave antennas in order to direct the emitted microwave radiation onto the desired target area. Triangulation represents a relatively straightforward operation for modern computers, and the computer-assisted triangulation of three microwave antennas is considered to be within the ability of those skilled in the art.

In instances where the diseased or damaged tissue resides within the subject's body, such as within a breast or cranium, the microwave radiation can be applied percutaneously, i.e., through unbroken skin. Thus, the one or more microwave antennas for use in the current invention can be configured for delivering microwave radiation from outside the subject's body. In other instances, one or more microwave antennas can be delivered into or near the target tissue, and the microwave radiation emitted from the antenna and directed onto the adjacent tissue from within the subject's body. Thus, one or more of the microwave antennas can be configured for delivering microwave radiation within the subject's body, and to this end may comprise a probe. The probe can be a conventional microwave antenna catheter, or can comprise a fiber optic cable. In the former instance, the microwave radiation will be produced by and emitted from the catheter itself; in the latter instance, where the probe comprises a fiber optic cable, the microwave radiation will be produced by an external antenna and transported through the fiber optic cable into the subject and onto the target tissue. The fiber optic transportation of microwaves is effected by techniques known to those skilled in the art.

The present invention is also directed to systems providing microwave treatment of tissue. The inventive systems comprise a microwave radiation generator capable of generating microwave radiation characterized as having at least one frequency component in the range of from about 4 GHz to about 12 GHz, the generator being operatively connected to at least one antenna, each of the one or more antennas being capable of transmitting microwave radiation to a tissue region; and, a source of particles, the particles being capable of absorbing at least a portion of the transmitted microwave radiation, and being capable of being placed into or near the tissue region.

The microwave generator for use in the present systems can be selected from commercially-available generators and custom-built machines. Microwave generators are readily available from various commercial vendors, including microwave generators capable of generating microwaves in the C- and X-band frequencies. In particular embodiments, the microwave generator is capable of generating microwave radiation characterized as having at least one frequency component in the range of from about 5 GHz to about 9 GHz, from about 6 GHz to about 8 GHz, or to about 6.5 GHz to about 7.5 GHz. Preferred microwave generators are capable of operating at a power of about 10 W, although microwave generators capable of operating at powers less than or greater than 10 W can also be used, depending the preference of the user.

Various off-the-shelf antennas can be used to supply one or more of the recited at least one antenna. In preferred embodiments, each of the at least one antenna(s) is capable of transmitting focused microwave radiation. It is also preferred that each of the at least one antenna is capable of being adjusted in order to direct said focused microwave radiation onto a desired target area. In some embodiments of the current invention, the microwave generator is operatively connected to three antennas. Where three microwave antennas are used, the antennas can be configured to permit adjustment. In such instances, it will be possible to adjust the directionality of one or more of the antennas so that the microwave radiation emitted therefrom will be directed onto the desired target area, which allows the concentration or focusing of the microwave radiation onto the chosen situs. One method of adjusting the antennas so that each one is focused onto the target area is triangulation, which can be accomplished manually or with the assistance of a machine, such as a computer. The instant systems of providing microwave treatment can further comprise a computer, which can be used to assist triangulation, to acquire images, to process images, to display data (e.g., temperature data, depth data), or for other useful purposes.

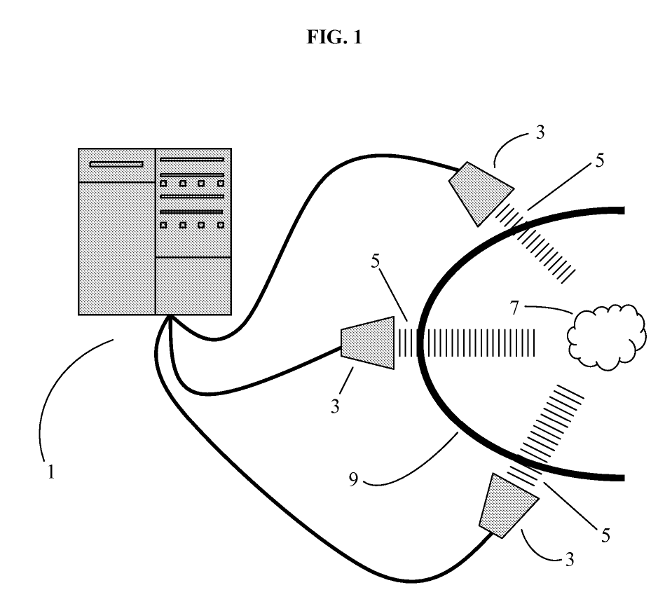

The one or more microwave antennas for use in the disclosed systems can be configured for delivering microwave radiation percutaneously from outside the subject's body. FIG. 1 depicts an exemplary system comprising a microwave array 1 having three antennas 3 that is used to percutaneously direct microwave energy 5 onto a preselected tissue region 7 within a subject's breast 9. In other instances, one or more microwave antennas can be configured for delivery into or near the target tissue, so that the microwave radiation emitted from the antenna and directed onto the adjacent tissue occurs from within the subject's body. For example, the antennas may constitute an adaptive phased array capable of delivering concentrated microwave energy to a desired point in space, and one or more of the antennas of the array may be configured for insertion beneath the subject's skin. Thus, in some embodiments, one or more of the microwave antennas can be configured for delivering microwave radiation within the subject's body, and to this end may comprise a probe. The probe can be a conventional microwave antenna catheter, or can comprise a fiber optic cable. In the former instance, the microwave radiation will be produced by and emitted from the catheter itself; in the latter instance, where the probe comprises a fiber optic cable, the microwave radiation will be produced by an external antenna and transported through the fiber optic cable into the subject and onto the target tissue. Fiber optic equipment and its combination with electromagnetic radiation-generating machinery is widely understood by those skilled in the art.

The inventive systems can further comprise a temperature probe for placement into or near the target tissue, so that the change in the tissue's temperature during microwave irradiation can be monitored, and the irradiation discontinued once the desired temperature change has been attained. The temperature probe may be configured for subcutaneous introduction into or near the tissue, or may make use of temperature-monitoring techniques that do not require subcutaneous insertion. The temperature probe may comprise an infrared device, or any other means of monitoring tissue temperature either from within the subject adjacent to or within the target tissue, or outside of the subject. Temperature-monitoring devices for use as the recited temperature probe are readily available to those skilled in the art.

In the present systems, the particles can comprise any material that is capable of absorbing at least a portion of the transmitted microwave radiation generated by the microwave generator. In preferred embodiments the material comprises carbon. The particles comprising carbon are preferably carbon substances that have a resonating microwave frequency of from about 4 GHz to about 12 GHz. Many forms of carbon are known by those skilled in the art, and, while not intending to exclude other carbon types, it is contemplated that any form of carbon having a resonating microwave frequency of from about 4 GHz to about 12 GHz will be within the scope of the present invention. For example, the particles comprising carbon can comprise carbon black. As discussed above, carbon blacks have chemisorbed oxygen complexes (e.g., carboxylic, quinonic, lactonic, phenolic groups and others) on their surfaces to varying degrees depending on the conditions of manufacture. These surface oxygen groups are collectively referred to as the volatile content. In preferred embodiments, the present invention uses carbon black having a moderate volatile content, for example, having about C20 or about C30 volatile constituents. Carbon blacks having a different volatile content are also contemplated as being within the scope of the present invention.

The constituent parts of the particles preferably have characteristic dimensions in the micrometer range, although other particle or fragment sizes may also be used. Because carbon particles or particles comprising another resonating material for use in the present invention can be present in numerous configurations, and can be irregular in shape, the term “characteristic dimensions” is used herein to describe the long axis in the case of substantially cylindrical or otherwise oblong particles, and to describe diameter in the case of substantially spherical particles, etc. In preferred embodiments wherein the wherein the carbon particles comprise carbon black, the particles can have characteristic dimensions of about 100 μm. In other embodiments, the particles can have characteristic dimensions of about 10 nm to about 500 μm, of about 100 nm to about 100 μm, or of about 200 nm to about 10 μm.

Preferred are particles having characteristic dimensions that are conducive to ready dispersion within delivery media. Although the particles can be contacted with the tissue by directly introducing the particles into the tissue environment, a more preferred method involves mixing the materials into a delivery medium, which is itself introduced into the tissue environment and contacted with the target tissue. Suitable delivery media can be liquid, in the form of solutions, suspensions, emulsions, syrups, elixirs, and the like. The selected delivery medium should be compatible with the human internal corporeal environment, and to this end aqueous solutions are a preferred delivery medium, one highly favored example being saline. As previously provided, it has been discovered that carbon black particles are readily dispersed within a saline delivery medium, which in turn is readily introduced into the human body. Carbon particles can be suitably dispersed in an aqueous saline medium using any of a variety of dispensing agents known to those skilled in the pigment dispersion art.

The inventive systems for microwave treatment of tissue can further comprise a delivery tool for delivering the particles into or near said tissue region. Preferred embodiments of the present systems are conducive to minimally invasive treatment, and the delivery tool is ideally compatible with treatment regimes that are minimally invasive, that can be conducted without general anesthesia, and that can be performed on an outpatient basis. Because the combination of the delivery medium and the particles will preferably take the form of a liquid solution, particles can be delivered through a needle via hypodermic injection. Thus, the delivery tool preferably comprises an injection tool for injecting the particles into or near tissue region that has been selected for treatment. Ideally, the injection tool is such that discomfort to the subject is minimized and the particles are delivered as precisely as possible. A high-gauge hypodermic needle represents a preferred delivery tool.

Prior to microwave irradiation of the tissue, or even prior to contacting the tissue with the carbon particles or other particles having a resonating frequency corresponding to the applied microwave radiation frequency, practitioners may wish to localize a target area of the tissue, so that, for example, a specific area of the tissue can be designated for contacting with resonating material, post-contacting irradiation, or both. The disclosed systems may therefore further comprise an imaging device for identifying a target area of the tissue, or to visualize some other feature of interest. Once a target area of the tissue has been identified, it can be contacted with resonating material that is precisely delivered into or near the identified area, or subjected to focused microwave radiation that has been directed particularly to that area. The target area, which can be a tumor mass or any other area of particular interest, can be identified using an imaging device. The imaging device may be selected from any suitable apparatus, including an ultrasound device, a magnetic resonance imaging device, a fluoroscopy device, a positron emission tomography device, a tomography device, a radiological device, and the like. Those skilled in the art will be familiar with the acquisition and use of such imaging devices.

### EXAMPLES

The present invention is further defined in the following Examples. It should be understood that these examples, while indicating preferred embodiments of the invention, are given by way of illustration only, and should not be construed as limiting the appended claims From the above discussion and these examples, one skilled in the art can ascertain the essential characteristics of this invention, and without departing from the spirit and scope thereof, can make various changes and modifications of the invention to adapt it to various usages and conditions.

### Example 1

### Method for Microwave Treatment of Breast Cancer Tumor

A female subject diagnosed as having an early-stage tumor in the right breast is selected for microwave treatment. Magnetic resonance imaging is used to locate the margins of the tumor in three-dimensonal space within the breast. Sterilized carbon black (100 μm, with a volatile content of 20 carbons) is dispersed in 50 cc sterile saline in an amount resulting in 40 weight percent carbon black within a saline/carbon black solution. The solution is stirred for several minutes until uniform disperson of the carbon black within the saline is observed. Thirty milliliters of the saline/carbon black is drawn into a hypodermic syringe using a sterile 25-gauge needle, and air bubbles are expelled from the syringe. A point of injection on the skin of the right breast is chosen based upon the closest point on the skin to the underlying tumor, and the point is wiped with an ethanol swab. The application of local anesthetic to the breast is optional. The needle is inserted at an angle and to a depth corresponding to the approximate center of the tumor as determined by the previous MRI scan. The entire 30 cc complement of the saline/carbon black mixture is injected into the tumor, and the needle is withdrawn. The subject is instructed to return to the clinic after two hours have elapsed, which allows time for the carbon black particles to disperse within the tumor.

Upon the subject's return to the clinic, the subject is seated on a comfortable, stationary bench and instructed to remain motionless during treatment, and the right breast is caused to rest on a flat treatment surface that is located in front of the subject. A microwave antenna array operatively attached to a microwave generator and comprising three antennas are positioned so that one antenna is located above the breast, and the remaining antennas are located to either side of the breast. The microwave generator produces 10 W of power and is capable of emitting radiation having a frequency that corresponds to the resonating frequency of the carbon black. Using computer-assisted triangulation and based on the MRI images obtained previously, the antennas are adjusted so that the microwave radiation that is emitted from the antennas will be focused onto the location of the tumor. A temperature probe is inserted into the breast by means of a small-diameter catheter until it reaches the approximate center of the tumor. A local anaesthetic is optional. The microwave generator is switched on, and the antennas begin to emit microwave radiation at a prescribed frequency of 6.8 GHz. During irradiation, the temperature of the tissue is carefully monitored using the readouts from the temperature probe. The subject is asked to remain as stationary as possible for the duration of the procedure. A temperature of approximately 108.6° F. is observed after 30 minutes at which time the microwave generator is deactivated. The temperature probe is removed, and the site at which the probe was inserted is closed.

Tumor measurements are taken in a subsequent visit that occurs one week after the episode of microwave therapy. Magnetic imaging is used to assess the extent of tumor necrosis and to assess whether additional treatments are necessary and, if so, where the microwave radiation should be focused.

When ranges are used herein for physical properties, such as molecular weight, or chemical properties, such as chemical formulae, all combinations, and subcombinations of ranges for specific embodiments therein are intended to be included.

The disclosures of each patent, patent application, and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A method for microwave treatment of tissue comprising:
contacting said tissue with particles comprising carbon; and,
subjecting said tissue to microwave radiation.

2. The method according to claim 1, wherein said microwave radiation is a pre-selected microwave radiation frequency.

3. The method according to claim 2, wherein said pre-selected microwave radiation frequency is characterized as having at least one frequency component in the range of from about 4 GHz to about 12 GHz.

4. The method according to claim 2, wherein said pre-selected microwave radiation frequency is characterized as having at least one frequency component in the range of from about 5 GHz to about 9 GHz.

5. The method according to claim 2, wherein said pre-selected microwave radiation frequency is characterized as having at least one frequency component in the range of from about 6 GHz to about 8 GHz.

6. The method according to claim 2, wherein said pre-selected microwave radiation frequency is characterized as having at least one frequency component in the range of from about 6.5 GHz and about 7.5 GHz.

7. The method according to claim 1 wherein said particles comprising carbon comprise carbon black.

8. The method according to claim 7 wherein said carbon black comprises particles having characteristic dimensions in the range of from about 10 nm to about 500 μm.

9. The method according to claim 1 wherein said contacting comprises injecting said particles comprising carbon into or near said tissue.

10. The method according to claim 1 wherein said subjecting said tissue to microwave radiation occurs for a time sufficient to raise the temperature of said tissue by at least about 5° F. above the temperature of said tissue prior to subjecting said tissue to microwave radiation.

11. The method according to claim 1 wherein said subjecting said tissue to microwave radiation occurs for a time sufficient to raise the temperature of said tissue by at least about 10° F. above the temperature of said tissue prior to subjecting said tissue to microwave radiation.

12. The method according to claim 1 wherein said subjecting said tissue to microwave radiation occurs for a time sufficient to raise the temperature of said tissue up to about 10° F. above the temperature of said tissue prior to subjecting said tissue to microwave radiation.

13. The method according to claim 1 wherein said microwave radiation is delivered percutaneously.

14. The method according to claim 1 wherein said microwave radiation is delivered using one or more microwave antennas.

15. The method of claim 14 wherein at least one of said one or more microwave antennas comprises a probe.

16. The method of said claim 15 further comprising delivering said at least one of said one or more microwave antennas into or near said tissue.

17. The method according to claim 14 wherein said microwave radiation is delivered using a plurality of microwave antennas.

18. The method according to claim 17 wherein said microwave radiation is delivered using three microwave antennas.

19. The method according to claim 18 wherein said microwave radiation from each of said three microwave antennas is directed onto a desired target area.

20. The method according to claim 18 further comprising adjusting one or more of said three microwave antennas to direct said microwave radiation onto a desired target area.

21. The method according to claim 1 further comprising identifying a target area of said tissue.

22. The method according to claim 21 wherein said target area is identified using an imaging device.

23. The method according to claim 22 wherein said imaging device is selected from an ultrasound device, a magnetic resonance imaging device, a fluoroscopy device, a positron emission tomography device, a tomography device, and a radiological device.

24. The method according to claim 1 wherein said tissue comprises damaged or diseased tissue.

25. The method according to claim 1 wherein said tissue comprises cancerous or precancerous tissue.

26. A method for microwave treatment of tissue comprising:
contacting said tissue with material having a resonating frequency in the range of from about 4 GHz to about 12 GHz; and,
subjecting said tissue to microwave radiation characterized as having at least one frequency component that corresponds to the resonating frequency of said material.

27. The method according to claim 26, wherein said microwave radiation frequency is characterized as having at least one frequency component in the range of from about 4 GHz to about 12 GHz.

28. The method according to claim 26, wherein said pre-selected microwave radiation frequency is characterized as having at least one frequency component in the range of from about 5 GHz to about 9 GHz.

29. The method according to claim 26, wherein said pre-selected microwave radiation frequency is characterized as having at least one frequency component in the range of from about 6 GHz to about 8 GHz.

30. The method according to claim 26, wherein said pre-selected microwave radiation frequency is characterized as having at least one frequency component in the range of from about 6.5 GHz and about 7.5 GHz.

31. The method according to claim 26 wherein said material comprises carbon.

32. The method according to claim 31 wherein said carbon comprises carbon black.

33. The method according to claim 32 wherein said carbon black comprises particles having characteristic dimensions in the range of from about 10 nm to about 500 μm.

34. The method according to claim 26 wherein said contacting comprises injecting said particles comprising carbon into or near said tissue.

35. The method according to claim 26 wherein said subjecting said tissue to microwave radiation occurs for a time sufficient to raise the temperature of said tissue by at least about 5° F. above the temperature of said tissue prior to subjecting said tissue to microwave radiation.

36. The method according to claim 26 wherein said subjecting said tissue to microwave radiation occurs for a time sufficient to raise the temperature of said tissue by at least about 10° F. above the temperature of said tissue prior to subjecting said tissue to microwave radiation.

37. The method according to claim 26 wherein said subjecting said tissue to microwave radiation occurs for a time sufficient to raise the temperature of said tissue up to about 10° F. above the temperature of said tissue prior to subjecting said tissue to microwave radiation.

38. The method according to claim 26 wherein said microwave radiation is delivered percutaneously.

39. The method according to claim 26 wherein said microwave radiation is delivered using one or more microwave antennas.

40. The method of claim 39 wherein at least one of said one or more microwave antennas comprises a probe.

41. The method of said claim 40 further comprising delivering said at least one of said one or more microwave antennas into or near said tissue.

42. The method according to claim 39 wherein said microwave radiation is delivered using a plurality of microwave antennas.

43. The method according to claim 42 wherein said microwave radiation is delivered using three microwave antennas.

44. The method according to claim 43 wherein said microwave radiation from each of said three microwave antennas is directed onto a desired target area.

45. The method according to claim 43 further comprising adjusting one or more of said three microwave antennas to direct said microwave radiation onto a desired target area.

46. The method according to claim 26 further comprising identifying a target area of said tissue.

47. The method according to claim 46 wherein said target area is identified using an imaging device.

48. The method according to claim 47 wherein said imaging device is selected from an ultrasound device, a magnetic resonance imaging device, a fluoroscopy device, a positron emission tomography device, a tomography device, and a radiological device.

49. The method according to claim 26 wherein said tissue comprises damaged or diseased tissue.

50. The method according to claim 26 wherein said tissue comprises cancerous or precancerous tissue.

51. A system for microwave treatment of tissue comprising:
a microwave radiation generator capable of generating microwave radiation characterized as having at least one frequency component in the range of from about 4 GHz to about 12 GHz, said generator being operatively connected to at least one antenna, each of said at least one antenna being capable of transmitting microwave radiation to a tissue region; and,
a source of particles, said particles being capable of absorbing at least a portion of the transmitted microwave radiation, and being capable of being placed into or near said tissue region.

52. The system according to claim 51 wherein each of said at least one antenna is capable of transmitting focused microwave radiation.

53. The system according to claim 52 wherein each of said at least one antenna is capable of being adjusted in order to direct said focused microwave radiation from each of said at least one antenna onto a desired target area.

54. The system according to claim 52 wherein said generator is operatively connected to three antennas.

55. The system according to claim 54 wherein each of said three antennas are capable of being adjusted in order to direct said focused microwave radiation from each of said three antennas onto a desired target area.

56. The system according to claim 52 wherein said at least one antenna comprises at least one probe.

57. The system according to claim 51, wherein said generator is capable of generating microwave radiation characterized as having at least one frequency component in the range of from about 5 GHz to about 9 GHz.

58. The system according to claim 51, wherein said generator is capable of generating microwave radiation characterized as having at least one frequency component in the range of from about 6 GHz to about 8 GHz.

59. The system according to claim 51, wherein said generator is capable of generating microwave radiation characterized as having at least one frequency component in the range of from about 6.5 GHz to about 7.5 GHz.

60. The system according to claim 51 wherein said particles comprise carbon.

61. The system according to claim 60 wherein said particles comprise carbon black.

62. The system according to claim 60 wherein said carbon black comprises particles having characteristic dimensions in the range of from about 10 nm to about 500 μm.

63. The system according to claim 51 further comprising a delivery tool for delivering said particles into or near said tissue region.

64. The system according to claim 63 wherein said delivery tool comprises an injection tool for injecting said particles into or near said tissue region.

65. The system according to claim 51 further comprising a delivery medium for delivering said particles into or near said tissue region.

66. The system according to claim 52 wherein one or more of said at least one antenna is configured to deliver said microwave radiation percutaneously.

67. The system according to claim 51 further comprising an imaging device.

68. The system according to claim 67 wherein said imaging device is selected from an ultrasound device, a magnetic resonance imaging device, a fluoroscopy device, a positron emission tomography device, a tomography device, and a radiological device.

69. The system according to claim 51 further comprising a temperature probe.
